(19) United States
(12) Patent Application Publication
(10) Pub. No.: US 2006/0041019 A1
(43) Pub. Date: Feb. 23, 2006

(54) Inhibitor of anticancer drug side effect

(76) Inventors: Yasukazu Nagato, Atsugi-shi (JP); Masahiro Murakami, Osaka-shi (JP)

Correspondence Address: GREENBLUM & BERNSTEIN, P.L.C., 1950 ROLAND CLARKE PLACE, RESTON, VA, 20191, US

(21) Appl. No.: 10/516,313

(22) PCT Filed: Jun. 12, 2003
(86) PCT No.: PCT/JP03/07478

§ 371 Date: Jul. 25, 2005

(30) Foreign Application Priority Data
Jun. 12, 2002 (JP) 2002-170781

(51) Int. Cl. A61K 31/225

(52) U.S. Cl. 514/547

(57) ABSTRACT

An object of the present invention is to provide a novel agent for suppressing side effects of an antitumor agent, which can suppress side effects caused by the use of an antitumor agent, such as hair loss. The present invention provides an agent for suppressing side effects of an antitumor agent which comprises a mixture of cyclic and/or straight chain poly lactic acids having a condensation degree of 3 to 20.

### TECHNICAL FIELD

The present invention relates to an agent for suppressing side effects of an antitumor agent and an agent for inhibiting hair loss. More particularly, the present invention relates to an agent that can suppress side effects of antitumor agent such as adriamycin, and can be used for a pharmaceutical preparation, food for specified health use, health food and the like capable of inhibiting hair loss.

### BACKGROUND ART

Recently, a wide variety of antitumor agents have been developed, and administration of antitumor agents is a major means for treating a solid tumors. Disadvantageously, many antitumor agents do not specifically and selectively affect tumor cells, but they also affect normal cells and produce side effects. The effectiveness of antitumor agents is enhanced via, for example, a multiple-drug therapy or short-term megadoses, and such techniques are widely applied in clinical settings. However, the issue of side effects resulting from the increased dosage is also a serious concern.

For example, a variety of antitumor agents are currently used in clinical settings. Examples thereof include: alkylating agents, such as nitrogen mustards and cyclophosphamide; antimetabolites, such as 5-fluorouracil and cytosine arabinoside; antibiotics, such as mitomycin and bleomycin; plant alkaloids; cisplatin; and hormone preparations. Side effects thereof, such as myelosuppression, hair loss, vomition, digestive tract disorders, hepatotoxicity, nephrotoxicity, cardiotoxicity, pulmonary toxicity, stomatitis, dermatopathy, or neurotoxicity, affect most of the body.

Hair loss is a side effect the severity of which significantly increases in proportion to the dosage of an antitumor agent and the intervals of administration. Although hair loss does not directly affect a patient's life and does not inflict physical pain upon a patient, the influence thereof on the patient's psychological condition is significant, and it is a serious problem that causes the quality of life (QOL) of the patient to deteriorate.

Human body hair grows via differentiation of hair matrix cells in the hair follicles in the body. The hair follicles on the head (the hair organ on the scalp) have the fastest growth rate and the growth period thereof is long. Accordingly, it is known that hair on the head has the property of growing the longest among all forms of body hair, and that the number of the hair follicles in the growth stage is large. Simply, alopecia is clinically classified as follows: male pattern alopecia; alopecia areata; senile alopecia; congenital alopecia; alopecia accompanying metabolic disorders such as endocrine abnormalities or systemic diseases such as malnutrition, shock, or persistent hyperthermia; secondary alopecia that follows diseases such as a variety of cutaneous symptoms that occur in the head hair; and drug alopecia. The causes thereof range from genetic factors to diseases, and alopecia would damage the hair follicles (the hair organ on the scalp) on the head. The mechanism of alopecia caused by antitumor agents has not yet been elucidated. Due to the significantly higher biological activity of the hair organ on the scalp compared with that of the hair organs at other locations, the hair organ on the scalp is susceptible to antitumor agents as are the bone marrow lymphoid tissue and the mucosal epithelium of the digestive tract, and the hair matrix cells in the hair follicles are damaged. Consequently, the growth of the hair matrix cell functions is interrupted, the hair bulb is deformed, and hair falls out in the form of atrophic hair or trichodystrophy. Alternatively, the hair organ rapidly moves to the resting stage and hair falls out.

Alopecia induced by antitumor agents frequently occurs with the use of, for example, anthracycline agents, including adriamycin, endoxan, or etoposide, and the severity of alopecia is high with the use of such agents. Such phenomena are also observed with the use of nitrosourea, 5-fluorouracil, cisplatin, interferon, and the like.

Examples of methods for inhibiting hair loss that is a side effect caused by an antitumor agent include: a method wherein an antitumor agent is administered in combination with an antagonist that is specific thereto (e.g., a method wherein Co-enzyme Q¹⁰ is used in combination); alteration of the route of administration in order to reduce the amount of the antitumor agent reaching the hair organ on the scalp by avoiding oral or intravenous administration (e.g., intraarterial or intraperitoneal administration); and a technique of blocking the blood flow to the scalp wherein the blood flow to the scalp is reduced with the use of tourniquets to thereby inhibit the administered antitumor agent from reaching the hair root. However, none of these techniques have provided sufficient effects to date. Concerning the alteration of the route of administration, intraarterial administration can be only applied to types of cancer, such as hepatic tumors, involving an obvious artery territory. The technique of blocking the blood flow to the scalp disadvantageously inflicts a great deal of pain upon a patient. Also available is a technique of cooling the cranium (the head), wherein alopecia is prevented by maintaining the scalp temperature at 22° C. or lower, but the evaluation of the effects attained thereby has not yet been confirmed. There is a report that this technique is absolutely ineffective in the case of oral administration, particularly when antitumor agent dosage is increased. Due to the necessity of cooling of the head for a long period of time, disadvantageously, the movement of a patient is restrained during such time, the patient feels uncomfortable due to his or her appearance, and a caregiver is required to undertake cumbersome tasks. Given these circumstances, development of an agent for suppressing side effects of an antitumor agent that can be safely used in order to improve the QOL of the patient during the continued administration of antitumor agents has been awaited.

In previous research, a mixture of cyclic and/or straight chain poly lactic acids having a condensation degree of 3 to 20 was reported to be useful as an anti-malignant tumor agent (JP Patent Publication (Kokai) Nos. 9-227388 A (1997) and 10-130153 A (1998)). However, the effects of the mixture of cyclic and/or straight chain poly lactic acids having a condensation degree of 3 to 20 on hair loss induced as a side effect of an antitumor agent have not yet been reported.

### DISCLOSURE OF THE INVENTION

An object of the present invention is to provide a novel agent for suppressing side effects of an antitumor agent, which can suppress side effects caused by the use of an antitumor agent, such as hair loss. It is another object of the present invention to provide a novel agent for inhibiting hair loss. It is still another object of the present invention to provide food and drink for suppressing side effects of an antitumor agent and for inhibiting hair loss utilizing the aforementioned agent.

In order to attain the above objects, the present inventors have studied the effects of a mixture of poly lactic acids on suppression of side effects of adriamycin by administering a mixture of cyclic and/or straight chain poly lactic acids having a condensation degree of 3 to 20 in combination with adriamycin to a mouse model of cancer. As a result, the aforementioned mixture of poly lactic acids was found to inhibit hair loss caused as a side effect of adriamycin. The present invention has been completed based on such findings.

Specifically, the present invention provides an agent for suppressing side effects of an antitumor agent which comprises a mixture of cyclic and/or straight chain poly lactic acids having a condensation degree of 3 to 20.

Another aspect of the present invention provides an agent for inhibiting hair loss which comprises a mixture of cyclic and/or straight chain poly lactic acids having a condensation degree of 3 to 20.

The agent according to the present invention can be preferably used for inhibiting hair loss caused by the use of antitumor agents.

Preferably, the lactic acid, which is a repeating unit in polylactic acid, is substantially comprised of L-lactic acid.

Preferably, the mixture of cyclic and/or straight chain poly lactic acids having a condensation degree of 3 to 20 is a mixture of polylactic acids that is produced by polymerizing lactide in the presence of the compound represented by formula (3): Me-N(R¹)(R²) wherein Me represents an alkali metal and R¹ and R² each independently represent an aliphatic group or an aromatic group.

Preferably, Me represents lithium, and R¹ and R² each independently represent an alkyl group having 1 to 6 carbon atoms in the above formula. More preferably, Me represents lithium, and R¹ and R² represent an isopropyl group in the above formula.

Preferably, the mixture of cyclic and/or straight chain poly lactic acids having a condensation degree of 3 to 20 is a substantially cyclic polylactic acid mixture.

Another aspect of the present invention provides food and drink for suppressing the side effects of an antitumor agent or inhibiting hair loss, which comprise the aforementioned agent for suppressing side effects of an antitumor agent or agent for inhibiting hair loss according to the present invention.

A further aspect of the present invention provides the use of a mixture of cyclic and/or straight chain poly lactic acids having a condensation degree of 3 to 20 in the production of the agent for suppressing side effects of an antitumor agent, an agent for inhibiting hair loss, and food and drink comprising the same.

A still further aspect of the present invention provides a method for suppressing side effects of an antitumor agent and/or inhibiting hair loss, which comprises administering an effective amount of a mixture of cyclic and/or straight chain poly lactic acids having a condensation degree of 3 to 20 to a mammal such as a human.

### BRIEF DESCRIPTION OF THE DRAWINGS

FIG. 1 is an overall view showing the FABMS spectrum (positive mode) of the product obtained in Production Example 1. Range: m/z 10.0000 to 1305.5900

FIG. 2 is an overall view showing the FABMS spectrum (negative mode) of the product obtained in Production Example 1. Range: m/z 10.0000 to 2000.0000

FIG. 3 is an enlarged view showing the FABMS spectrum (negative mode) of the product obtained in Production Example 1. Range: m/z 10,000 to 501.9260

FIG. 4 is an enlarged view showing the FABMS spectrum (negative mode) of the product obtained in Production Example 1. Range: m/z 490.2980 to 1003.7700

FIG. 5 is an enlarged view showing the FABMS spectrum (negative mode) of the product obtained in Production Example 1. Range: m/z 999.9500 to 1504.3400

FIG. 6 is an enlarged view showing the FABMS spectrum (negative mode) of the product obtained in Production Example 1. Range: m/z 1484.5300 to 2000.0000

FIG. 7 is an overall view showing the NMR spectrum of the product obtained in Production Example 1.

FIG. 8 shows the effects of CPL and adriamycin for suppressing hyperplasia.

FIG. 9 shows the results of comparison of the ranges of hyperplasia development among groups.

### BEST MODES FOR CARRYING OUT THE INVENTION

Hereafter, the embodiments of the present invention and the methods for carrying them out are described in detail.

The agent for suppressing side effects of an antitumor agent and the agent for inhibiting hair loss according to the present invention (which may be hereafter referred to as “the agents of the present invention”) comprise, as an active ingredient, a mixture of cyclic and/or straight chain poly lactic acids having a condensation degree of 3 to 20. They can be used, for example, for inhibiting hair loss that is caused as a side effect of an antitumor agent.

The term “side effects of an antitumor agent” used herein refers to all the unfavorable symptoms generated in the organism due to the administration of antitumor agents. Examples thereof include hair loss, myelosuppression, vomition, digestive tract disorders, hepatotoxicity, nephrotoxicity, cardiotoxicity, pulmonary toxicity, stomatitis, dermatopathy, and neurotoxicity. The agent for suppressing side effects of an antitumor agent according to the present invention can be used for inhibiting hair loss, among the aforementioned side effects.

Examples of antitumor agents, the side effects (e.g., hair loss) of which should be suppressed by administration of the mixture of poly lactic acids according to the present invention, include antibiotic antitumor agents, such as adriamycin, daunorubicin, daunomycin, aclacinomycin A, actinomycin D, mitomycin C, chromomycin A₃, bleomycin, peplomycin, neocarzinostatin, and auromomycin. Examples of other antitumor agents include: etoposide, which is the plant alkaloid podophyllin compound; other plant alkaloid antitumor agents, such as vincristine, vinblastine, and vindesin; antimetabolic antitumor agents, such as methotrexate, 5-fluorouracil, 5-fluorodeoxyuridine, tegafur, carmofur, cytosine arabinoside, cyclocytidine, 6-mercaptopurine, 6-mercaptopurine riboside, and 6-thioguanine; alkylating antitumor agents, such as nitrogen mustards, cyclophosphamide, nimustine, ranimustine, and carboquone; and other antitumor agents, such as L-asparaginase, cisplatin, estramustine, picibanil, krestin, lenthinan, schizophyllan, levamisole, bestatin, forphenicinol, and hormone preparations.

In the agent and food and drink of the present invention, a mixture of cyclic and/or straight chain poly lactic acids having a condensation degree of 3 to 20 is used as an active ingredient.

The term “a mixture of poly lactic acids” used in the present invention means a mixture wherein cyclic and/or straight chain poly lactic acids having a condensation degree of 3 to 20 are present at any ratio. That is to say, the term “mixture” does not only mean a mixture of poly lactic acids having any condensation degree ranging from 3 to 20, but is also used as a concept including a mixture of cyclic and straight chain poly lactic acids. As is described below in the present specification, “a mixture of poly lactic acids” can be obtained by condensing lactic acids by dehydration and then performing purification by a suitable method. Although the term “a mixture of poly lactic acids” is used in the present specification for the sake of convenience, this term also includes a poly lactic acid consisting of a single ingredient such as a cyclic poly lactic acid having single condensation degree or a straight chain poly lactic acid having single condensation degree.

The term “condensation degree” is used to mean the number of lactic acid unit that is a repeating unit in poly lactic acids. For example, the cyclic poly lactic acid is assumed to have the following structural formula wherein n represents condensation degree (n=3 to 20).

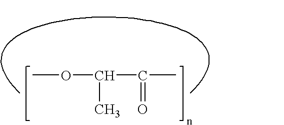

When “lactic acid” is simply referred to in the present specification, this lactic acid includes all of L-lactic acid, D-lactic acid or a mixture comprising these types of lactic acid at any ratio. Preferably in the present invention, the lactic acid consists substantially of L-lactic acid. The term “substantially” is used herein to mean that the ratio of L-lactic acid units in a mixture of poly lactic acids (number of L-lactic acid unit/number of L-lactic acid unit+number of D-lactic acid unit×100) is, for example, 70% or more, preferably 80% or more, more preferably 85% or more, further more preferably 90% or more, and particularly preferably 95% or more. The ratio of L-lactic acid units in a mixture of poly lactic acids depends on the ratio of L-lactic acid and D-lactic acid that exist in lactic acids used as a starting substance.

The methods for producing a mixture of cyclic and/or straight chain poly lactic acids having a condensation degree of 3 to 20 are not particularly limited, and the mixture of poly lactic acids can be obtained by the production methods described, for example, in Japanese Patent Application Laying-Open (Kokai) Nos. 9-227388 and 10-130153 or Japanese Patent Application No. 11-39894 (All publications cited herein are incorporated herein by reference in their entirety).

More specifically, for example, a mixture of cyclic and/or straight chain poly lactic acids having a condensation degree of 3 to 20 can be obtained by the following method A.

Method A:

First, lactic acid (preferably, lactic acid substantially consisting of L-lactic acid) is condensed by dehydration under an inactive atmosphere. Examples of the inactive atmosphere include nitrogen gas and argon gas, and nitrogen gas is preferred.

Dehydration and condensation reaction is carried out at a temperature of 110° C. to 210° C., preferably 130° C. to 190° C. under normal pressure to reduced pressure of approximately 1 mmHg, and particularly preferably the reaction is carried out by stepwise decompression and stepwise temperature rise. A reaction period can be determined as appropriate. For example, the reaction can be carried out for 1 to 20 hours. Where stepwise decompression and stepwise temperature rise are applied, reaction is performed by dividing the reaction period into two or more partial reaction periods, and then determining pressure and temperature for each of the reaction periods. Where stepwise decompression is applied, pressure can be reduced, for example, from a normal pressure to 150 mmHg and then to 3 mmHg. Where stepwise temperature rise is applied, temperature can be raised, for example, from 145° C. to 155° C. and then to 185° C. Practically, the reaction can be carried out by using these conditions in combination, for example, 145° C., normal pressure, 3 hours; 145° C., 150 mmHg, 3 hours; 155° C., 3 mmHg, 3 hours; and 185° C., 3 mmHg, 1.5 hours.

Subsequently, ethanol and methanol are added to the reaction mixture obtained by the dehydration and condensation reaction, and the mixture is filtered. The obtained filtrate is dried to obtain ethanol- and methanol-soluble fractions. The term “ethanol- and methanol-soluble fractions” is used in the present specification to mean fractions soluble in a mixed solution of ethanol and methanol. In order to obtain ethanol and methanol-soluble fractions, a reaction mixture obtained by dehydration and condensation reaction is mixed with ethanol and methanol, where the ratio of ethanol and methanol can be determined as appropriate. For example, the ratio is ethanol:methanol=1:9. The order, method and the like for adding ethanol and methanol to a reaction mixture are not limited, and may be selected as appropriate. For example, ethanol may be added at first to the reaction mixture obtained by the dehydration and condensation reaction, and then methanol may be added thereto.

The thus obtained ethanol- and methanol-soluble fractions are subjected to reverse phase column chromatography, especially to chromatography where an octadecylsilane (ODS) column is used. First, fractions eluted with 25 to 50 weight % acetonitrile aqueous solution of pH 2 to 3 are removed, and then fractions eluted with 90 weight % or more acetonitrile aqueous solution of pH 2 to 3, preferably 99 weight % or more acetonitrile aqueous solution, are collected so as to obtain a mixture of cyclic and/or straight chain poly lactic acids having a condensation degree of 3 to 20.

The thus obtained mixture of cyclic and/or straight chain poly lactic acids is neutralized with an alkaline substance such as sodium hydroxide, and is dried under reduced pressure, and then according to standard techniques, the mixture can be formulated in a desired form as mentioned below.

Other examples of the methods for producing a mixture of cyclic and/or straight chain poly lactic acids having a condensation degree of 3 to 20 used in the present invention include a method described in Japanese Patent Application No. 11-265715 (hereinafter referred to as method B), or a method described in Japanese Patent Application No. 11-265732 (hereinafter referred to as method C) (All publications cited herein are incorporated herein by reference in their entirety). Methods B and C will be described specifically below.

Method B:

Method B is a method for producing a cyclic lactic acid oligomer which comprises polymerizing lactid in the presence of a lithium compound represented by RYLi [wherein R represents an aliphatic group or aromatic group, Y represents oxygen atom or sulfur atom]. In the case of performing the polymerization reaction, the ratio of the amounts of the lithium compound (RYLi) is 1-0.1 mol, preferably 0.2-0.3 mol per mol of lactide. The reaction temperature is −100 to 0° C., preferably −78 to −50° C. Reaction is preferably carried out by starting from a temperature of −78 to −50° C. and gradually raising it to room temperature. The reaction is preferably carried out in the presence of a reaction solvent. As the reaction solvent, there can be used, for example, a cyclic ether such as tetrahydrofuran, diethylether, and dimethoxyethane. The reaction atmosphere can be an inactive gas atmosphere such as nitrogen gas and argon. The reaction pressure is not limited, and is preferably a normal pressure.

The composition (that is, the mixing ratio of cyclic lactic acid oligomer and a chain lactic acid oligomer) of the lactic acid oligomer obtained as described above fluctuates depending on the lithium compound used as a reaction assistant. Where a lithium compound of alkyl alcohol having a carbon number of 1 to 3 (ROLi) (wherein R represents an alkyl group with carbon number 1 to 3) is used as a lithium compound, a mixture of a cyclic lactic acid oligomer and a chain oligomer (proportion of the cyclic lactic acid oligomer: 80 to 85 weight %) is obtained. When a lithium compound of alkyl alcohol having a carbon number of 4 or more such as t-butyl alcohol, or thiophenol compound is used as a lithium compound, substantially only a cyclic lactic acid oligomer can be selectively obtained.

Method C:

This method comprises: (i) a first heating step which comprises heating lactic acid under a pressure condition of 350 to 400 mmHg and to a temperature of 120 to 140° C. so as to perform dehydration and condensation, and distilling off and removing only by-product water without distilling lactid off; (ii) a second heating step for synthesizing a product condensed by dehydration comprising chain lactic acid oligomers as the main ingredient, which comprises, after completion of the first heating step, heating the reaction product to a temperature of 150 to 160° C. while reducing the reaction pressure to 15 to 20 mmHg at a decompression rate of 0.5 to 1 mmHg/min, wherein only by-product water is distilled off and removed while avoiding distillation of lactid; and after the reaction pressure is reduced to 15 to 20 mmHg, maintaining the reaction under the same pressure condition and at a reaction temperature of 150 to 160° C.; (iii) a third heating step for synthesizing cyclic oligomers which comprises, after completion of the second heating step, heating under a pressure condition of 0.1 to 3 mmHg and at 150 to 160° C. to cyclize the chain lactic oligomer.

In this method, first, in the first heating step, lactic acid is heated under reduced pressure to perform dehydration and compression reaction. In this case the reaction period is 3 to 12 hours, preferably 5 to 6 hours. To allow the reaction in the first heating step to proceed smoothly, by-product water produced by condensation of lactic acids by dehydration is distilled off. At this time, distillation of by-product water is performed such that lactid, which is the dehydrated condensed product of two molecules of lactic acid, is not distilled off. To achieve such purpose, the reaction pressure is maintained at a reduced pressure, preferably 300 to 500 mmHg, more preferably 350 to 400 mmHg. Under this pressure condition, heating is performed at a temperature range of 100 to 140° C., preferably 130 to 140° C. The reaction product produced by reaction in the first heating step mainly comprises as the main ingredient a dehydrated condensed product of 3 to 23 molecules of lactic acid.

To obtain oligomers having an increased average degree of polymerization in the second heating step after completion of the above first heating step, heating is performed at a temperature higher than the reaction temperature of the above first heating step, preferably at 145° C. to 180° C., more preferably 150° C. to 160° C., while the reaction pressure is reduced to 10 to 50 mmHg, preferably 15 to 20 mmHg, so that dehydration and condensation reaction is further continued.

As with the reaction in the above first heating step, reaction is performed under a condition where by-product water, but not lactid, is distilled off, to allow the reaction to proceed smoothly. The rate at which reaction pressure is reduced to a pressure in the above range (decompression rate) is normally required to be maintained within a range of 0.25 to 5 mmHg/min, preferably 0.5 to 1 mmHg/min, in order to avoid distillation of lactid and increase the reaction efficiency. A decompression rate lower than the above range is not preferred because it will increase the time required to reduce pressure to a given pressure. On the other hand, a decompression rate higher than the above range is also not preferred because it will cause lactid to be distilled off together with by-product water.

After the reaction pressure is reduced to a certain pressure, reaction is further continued at that reaction pressure. The heating time period in this case is 3 to 12 hours, preferably 5 to 6 hours.

A lactic acid oligomer having an average polymerization degree of 3 to 30, preferably 3 to 23 is obtained by the reaction in the above second heating step. The proportion of cyclic oligomers in the oligomers in this case is normally about 70 to 80 weight %.

In the third heating step, after completion of the above second heating step, a reaction pressure is maintained at 0.25 to 5 mmHg, preferably 0.5 to 1 mmHg, and reaction is further continued at a temperature of 145 to 180° C., preferably 150 to 160° C. A reaction period is 3 to 12 hours, preferably 5 to 6 hours. By-product water produced in this case is also distilled off. In this case, distillation of lactid is preferably avoided. However, since the reaction product contains almost no lactid, it is not required to specially lower the decompression rate.

Lactic acid oligomers produced by reaction in the above third heating step have an average polymerization degree of 3 to 30, preferably 3 to 23, and contain cyclic oligomer in the proportion of 90 weight % or more, preferably 99 weight % or more.

Method D:

In a preferred embodiment of the present invention, the lactides are allowed to react in the presence of an alkali metal compound represented by the formula (3): Me-N(R¹)(R²). The formula (3): Me-N(R¹)(R²) is explained below.

In the formula (3), Me represents an alkali metal, and each of R¹ and R² independently represents an aliphatic group or aromatic group.

Examples of the aliphatic group defined in the present specification include a straight chain, branched chain, cyclic, or their combined form, saturated or unsaturated aliphatic hydrocarbon group containing 1 to 12, and preferably 1 to 6 carbon atoms. Specific examples include alkyl groups such as methyl, ethyl, n-propyl, i-propyl, n-butyl, i-butyl, t-butyl, octyl and dodecyl, and cycloalkyl groups such as cyclopropyl, cyclobutyl, cyclooctyl and cyclododecyl. The aliphatic group may be an unsaturated hydrocarbon group having a double or triple bond.

Examples of the aromatic group defined in the present invention include an aryl group and an arylalkyl group, containing 6 to 30, preferably 6 to 20, more preferably 6 to 12, and further more preferably 6 to 10 carbon atoms. Examples of the aryl group include phenyl, tolyl and naphthyl, and examples of the arylalkyl group include benzyl, phenethyl and naphthylmethyl.

The aliphatic group and the aromatic group may have one or more substituent(s). The type of substituents is not particularly limited, and the examples include a straight chain, branched chain, linear or cyclic alkyl group, a straight chain, branched chain, linear or cyclic alkenyl group, a straight chain, branched chain, linear or cyclic alkynyl group, an aryl group, an acyloxy group, an alkoxycarbonyloxy group, an aryloxycarbonyloxy group, a carbamoyloxy group, a carbonamide group, a sulfonamide group, a carbamoyl group, a sulfamoyl group, an alkoxy group, an aryloxy group, an aryloxycarbonyl group, an alkoxycarbonyl group, an N-acylsulfamoyl group, an N-sulfamoylcarbamoyl group, an alkylsulfonyl group, an arylsulfonyl group, an alkoxycarbonylamino group, an aryloxycarbonylamino group, an amino group, an ammonio group, a cyano group, a nitro group, a carboxyl group, a hydroxyl group, a sulfo group, a mercapto group, an alkylsulfinyl group, an arylsulfinyl group, an alkylthio group, an arylthio group, an ureide group, a heterocyclic group (e.g., a monocyclic or condensed ring containing at least one or more nitrogen, oxygen or sulfur atom(s) and consisting of 3 to 12 ring forming members), a heterocyclic oxy group, a heterocyclic thio group, an acyl group, a sulfamoylamino group, a silyl group, and a halogen atom. In the above, the carbon number of alkyl, alkenyl, alkynyl and alkoxy is generally 1 to 12, and preferably 1 to 6, and the carbon number of aryl is generally 6 to 20, and preferably 6 to 10.

In the formula (3), Me represents an alkali metal. Examples of an alkali metal include Li, Na and K, and Li is preferred.

Among the compounds represented by the formula (3), the compounds having asymmetric carbon atoms may be any one of (R) form, (S) form, and (R),(S) form.

A method for obtaining an alkali metal compound represented by the formula (3) is not particularly limited, and a person skilled in the art can obtain the compound as appropriate. For example, the alkali metal compound can be obtained by reaction of dialkylamine such as diisopropylamine with an alkylated alkali metal such as n-butyllithium. More specifically, this reaction can be carried out, for example, by mixing a solution containing dialkylamine in an inert solvent such as THF with a solution containing an alkylated alkali metal in an inert solvent such as hexane under conditions that are inactive for the reaction, e.g., under a nitrogen gas atmosphere, and then stirring the mixture. The reaction temperature is not particularly limited, as long as the reaction progresses, but it is preferably −78° C. to room temperature. The reaction temperature can be set as appropriate.

When lactides are polymerized in the presence of a compound represented by the formula (3), the used amount of the compound represented by the formula (3) (Me-N(R¹)(R²)) is preferably 0.1 to 1 mol, and more preferably 0.2 to 0.3 mol per mole of lactide.

When the polymerization reaction of lactides is carried out, the reaction temperature is not particularly limited as long as the reaction progresses, but it is preferably −100° C. to room temperature, and more preferably −78° C. to room temperature.

Polymerization reaction of lactides is preferably carried out in the presence of a reaction solvent. The reaction solvent is not particularly limited as long as it is inactive for the reaction. Examples of preferred solvents include cyclic ethers such as tetrahydrofuran, diethylether, and dimethoxyethane. Examples of reaction atmospheres to be used may include inactive gas atmospheres such as nitrogen gas and argon gas. Reaction pressure is not particularly limited, and it is preferably normal pressure.

The composition of the mixture of linear and cyclic lactic acid oligomers which is obtained by the method as mentioned above is altered depending on the type of the compound of the formula (3) used as a reaction assistant and the reaction conditions. Preferably, the content of linear lactic acid oligomer is higher than that of cyclic lactic acid oligomer.

According to the method as mentioned above, there is produced a mixture of linear and cyclic lactic acid oligomers represented by the following formula (1) or (2): 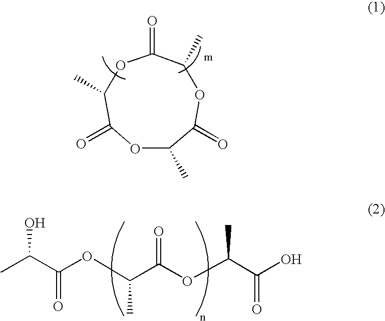
wherein m represents an integer of 1 to 18, and n represents an integer of 1 to 18.

The above methods A, B, C and D merely show some of specific examples of methods of producing a mixture of poly lactic acids used in the present invention. A mixture of poly lactic acids which is produced by other methods can also be used in the present invention.

When required, the agent of the present invention can be prepared by using, in addition to the essential component as described above, components or additives used for preparation of a dug such as medical drugs, quasi-drugs and the like by free selection and combination in a range which does not damage an effect of the invention. The agent of the present invention can be used by compounding it in medical drugs, quasi-drugs and the like in addition to a use as a single drug.

The form of the agent of the present invention is not specially restricted, but the appropriate form most suitable for a purpose can be selected from drug forms for oral administration or parenteral administration.

The drug preparation form suitable for oral administration includes, for example, a tablet, capsule, powder, drink, granule, fine granule, syrup, solution, emulsion, suspension, chewable and the like. The drug preparation form suitable for parenteral administration includes, for example, injection (subcutaneous injection, intramuscular injection, intravenous injection and the like), external use, drip, inhalation, spray and the like and, however, is not restricted to these forms.

A liquid drug preparation, for example, solution, emulsion or syrup, which is suitable for oral administration, can be prepared by using water, saccharides such as sucrose, sorbit and fructose, glycols such as polyethylene glycol and propylene glycol, oils such as sesame oil, olive oil and soybean oil, antiseptic agents such as p-hydroxy benzoic acid esters, and flavors such as strawberry flavor and peppermint. On the other hand, solid drug preparation, for example, tablet, capsule, powder, granule and the lice, can be prepared by using an excipient such as lactose, glucose, sucrose and mannite, a disintegrating agent such as starch and sodium alginate, a lubricant such as magnesium stearate and talc, a binder such as polyvinyl alcohol, hydroxy propyl cellulose and gelatin, a surfactant such as fatty acid ester, a plasticizer such as glycerin.

The drug preparation for injection or drip suitable for parenteral administration includes preferably the material, which is the active ingredient, as described above in a sterilized water-based medium, which is isotonic to blood of a recipient, in a dissolved or suspended condition. For example, in case of the injection, the solution can be prepared by using a water-based medium and the like composed of a salt solution, a glucose solution, or the mixture of the glucose solution with the salt solution. The drug preparation for intestinal administration can be prepared by using a carrier such as cacao butter, hydrogenated fat or hydrogenated carboxylic acid, and can be used as a suppository. In addition, for preparation of spray, a carrier which allows the material being the active ingredient as described above to disperse as fine particles, does not irritate a mouth cavity and an air way mucosa of the recipient, and makes absorption of the active ingredient easy, can be used. The carrier is specifically exemplified by lactic acid, glycerine and the like. In accordance with a property of the material being the active ingredient and the carrier to be used, the drug preparation having forms such as an aerosol or dry powder can be prepared. These preparations for parenteral administration are also added with 1 or 2 or more species of eatables and drinkables selected from glycols, oils, flavors, antiseptic agents, excipients, disintegrating agents, lubricants, binders, surfactants, plasticizers or the like.

The dose and administration frequency of the agent of the present invention can be properly selected in accordance with various factors including a purpose of administration, a form of administration, conditions of a recipient such as an age, body weight and sexuality and, however, as a rule, the amount of administration of the active ingredient ranges from 1 to 10,000 mg/kg/day, preferably 10 to 2,000 mg/kg/day, more preferably 10 to 200 mg/kg/day. It is preferable to administer the preparation of the amount as described above in 1 to 4 frequencies a day.

The timing of administration of the agent of the present invention is not particularly limited. When suppression of side effects of an antitumor agent is intended, the agent of the present invention may be administered before, during, or after the administration of the antitumor agent. Since the agent for inhibiting hair loss according to the present invention has the effect of inhibiting hair loss, it can be ingested not only at the time of administration of the antitumor agent but also on a routine basis in the form of health foods or pharmaceutical preparations.

The present invention also relates to food and drink for suppressing the side effects of an antitumor agent or inhibiting hair loss, which comprises a mixture of cyclic and/or straight chain poly lactic acids having a condensation degree of 3 to 20. Namely, the mixture of cyclic and/or straight chain poly lactic acids having a condensation degree of 3 to 20 which is used in the present invention can be not only used in the forms of a single preparation as described above, but also can be used by compounding it in food and drink.

A compounding form of food and drink according to the invention is not specially restricted, when it is satisfied to compound the mixture of poly lactic acids without decomposition.

The product of food and drink according to the present invention includes specifically a health food or a supplementary food including beverages, which is generally called a refreshing drink, drink agent, health food, specified health food, functional food, function activating food, nutriceutical food, supplement, feed, feed additive and the like.

Food and drink include arbitrary food and dinks, and examples thereof include confectionaries such as chewing gum, chocolate, candy, tablet confectionary, jelly, cookie, biscuit and yogurt, cold confectioneries such as ice cream and ice confectionary, beverages such as tea, refreshing drink (including juice, coffee, cocoa and the like), nourishment drink agent and esthetic drink agent, bread, ham, soup, jam, spaghetti, frozen foods. Alternatively, the mixture of poly lactic acids used in the present invention can also be used by adding it to a flavoring material or a food additive. By taking food and drink according to the present invention, the effect of suppressing side effects of an antitumor agent is obtained to provide safe food and drink which show no substantially harmful adverse effect.

The food and drink according to the present invention can be obtained by directly mixing and dispersing the mixture of poly lactic acids to a common material used for foods and then processing the same in a desired form by a publicly known method.

The food and drink according to the present invention encompasses food and drink in every form, and the types are not specifically limited. That is, the food and drink can be provided by mixing the agent for suppressing side effects of an antitumor agent according to the present invention into the above-mentioned various food and drink, or various nutrient compositions, such as various oral or enteral nutrient preparations or drinks. Compositions of such food and drink may include protein, lipid, carbohydrate, vitamin and/or mineral, in addition to the mixture of cyclic and/or straight chain poly lactic acids having a condensation degree of 3 to 20. The form of the food and drink is not specifically limited, and may be in any form, such as solid, powdery, liquid, gel, and slurry forms, so far as it is in a form that is easily ingested.

The content of the mixture of poly lactic acids in the food and drink is not specifically limited, and is generally 0.1 to 20 weight %, more preferably approximately 0.1 to 10 weight %.

The mixture of poly lactic acids is preferably contained in the food and drink in an amount which achieves an effect of suppressing side effects of an antitumor agent or inhibiting hair loss which is an object of the present invention. Preferably, about 0.1 g to 10 g, more preferably about 0.5 g to 3 g, of the mixture of poly lactic acids is contained per food or drink to be ingested.

The present invention is further described in the following examples, but the scope of the present invention is not limited by the examples in any way.

### EXAMPLE

### Production Example 1

### Production of a Mixture of Poly Lactic Acids (Hereinafter Referred to as CPL)

The reaction scheme of Production Example 1 is shown below.

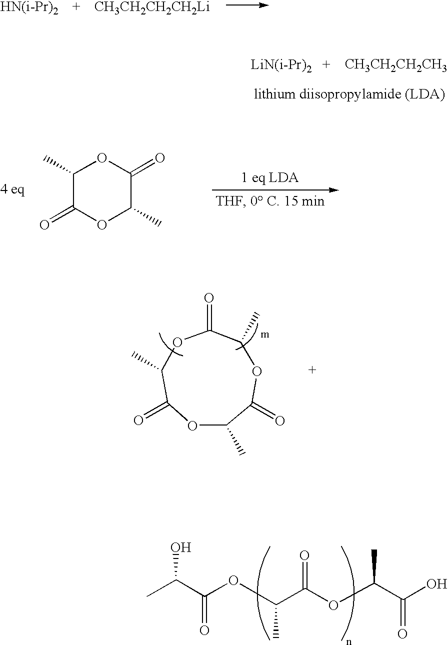

0.63 ml of n-butyllithium (1.6 M hexane solution, 1 mmol) was added to a 5 ml THF solution containing 0.101 g (1 mmol) of diisopropylamine at 0° C. under a nitrogen gas atmosphere, and the obtained mixture was stirred for 10 minutes, so as to obtain lithium diisopropylamide (LDA). Thereafter, 4 ml of THF solution containing 0.577 g (4 mmol) of L-(−)-lactide was added thereto followed by stirring for 15 minutes for reaction. Thereafter, 20 ml of a saturated ammonium chloride aqueous solution was added to the obtained reaction mixture to treat the reaction, and 10 ml of water was further added thereto. Extractions were carried out 5 times with THF (50 ml), and the organic layer was dried with anhydrous sodium sulfate. After anhydrous sodium sulfate was filtrated, the organic solvent was subjected to vacuum concentration, so as to obtain 0.53 g of a crude product. 6 ml of ether was added to the obtained crude product, and the mixture was immersed in an ultrasonic cleaner for 10 minutes for filtration, so as to obtain 0.39 g of a white solid product having a melting point of 125° C. to 129° C.

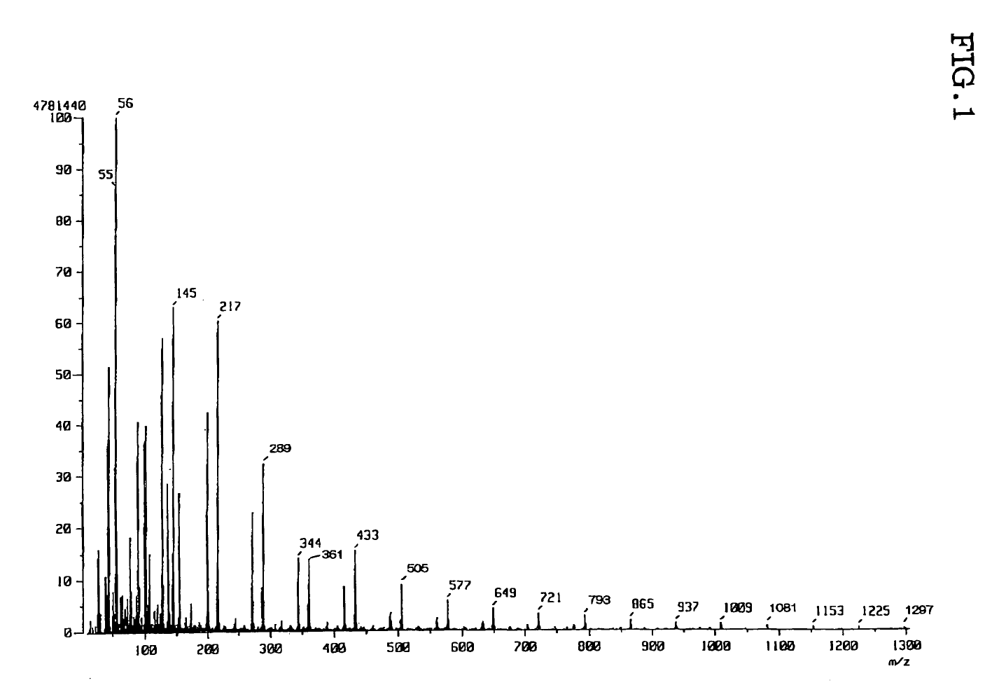

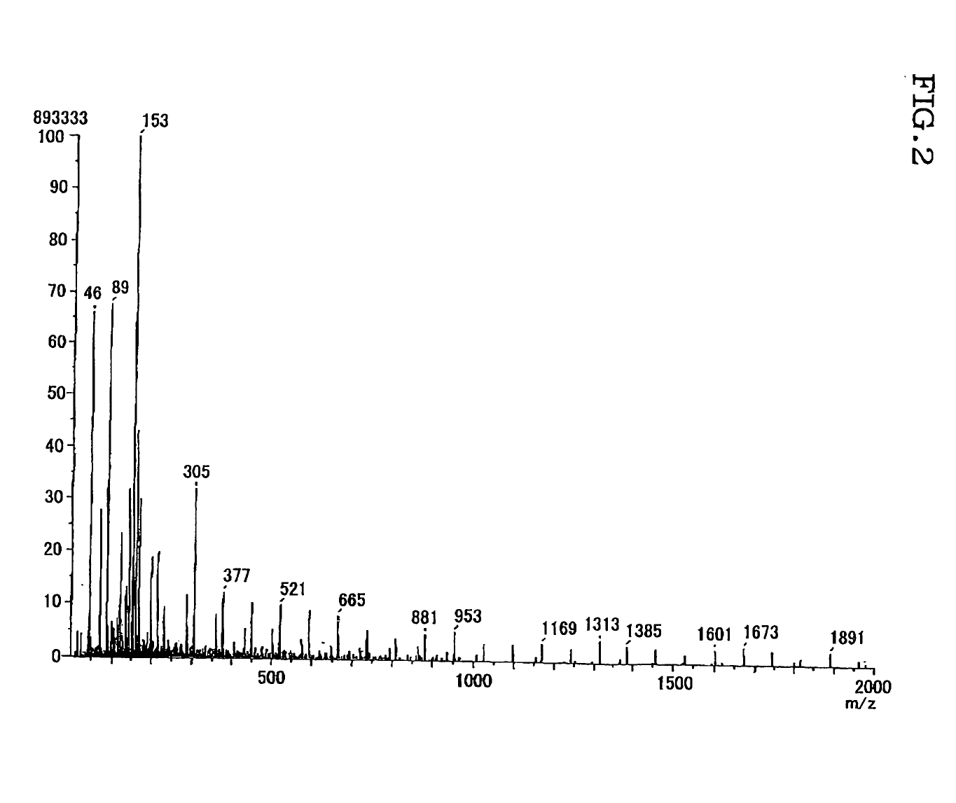

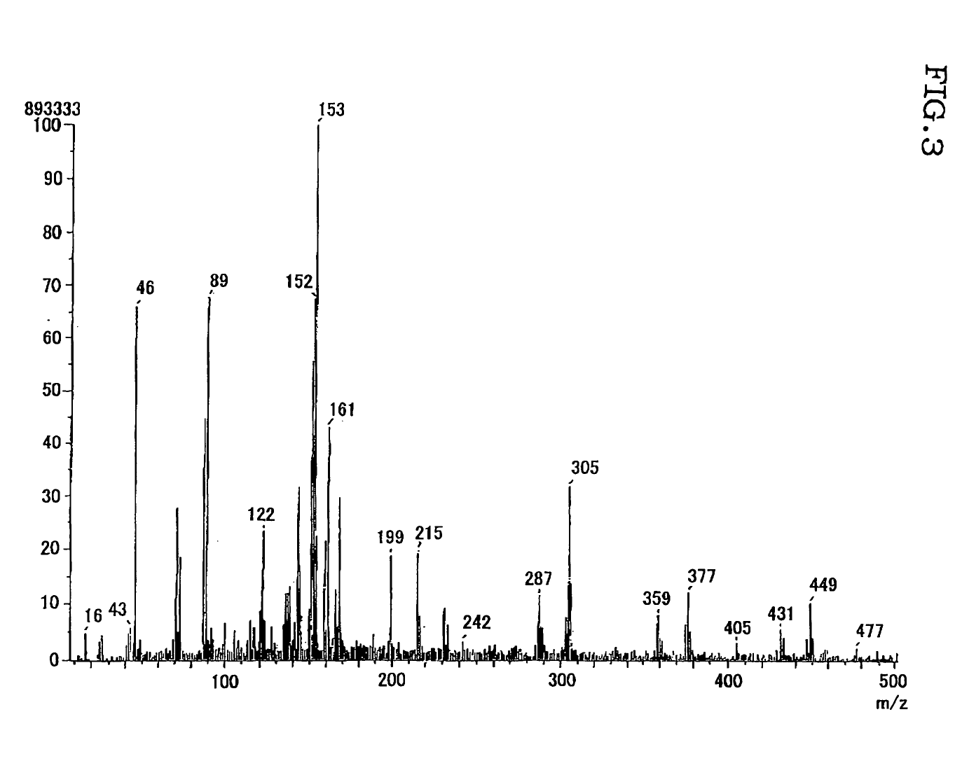

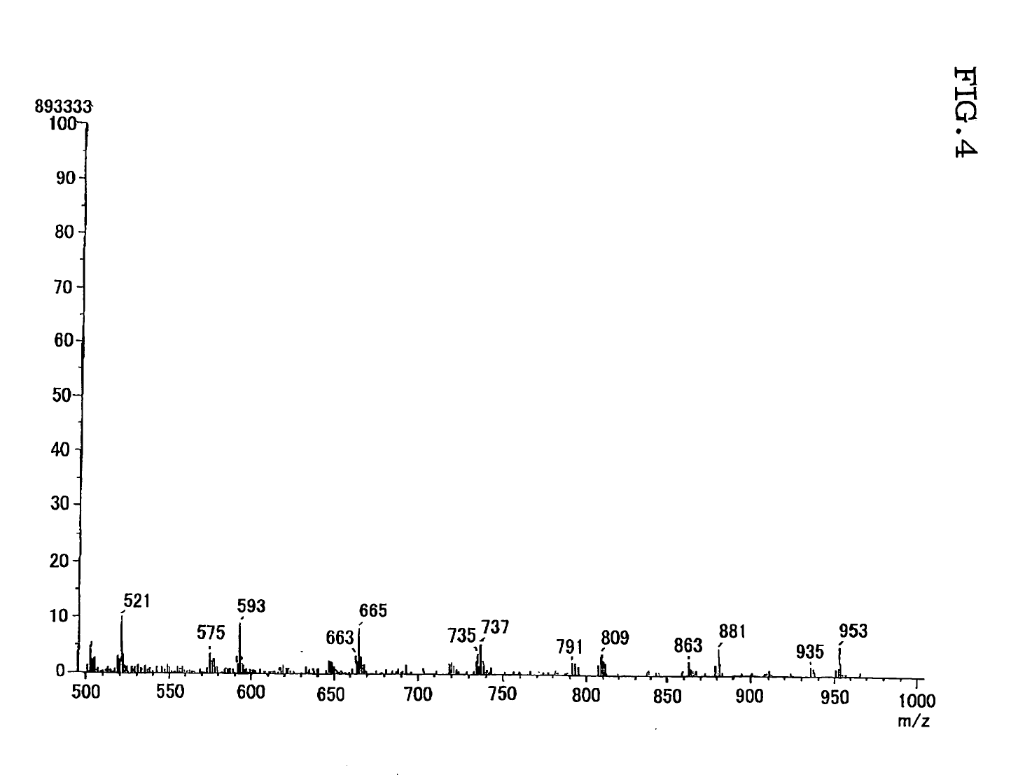

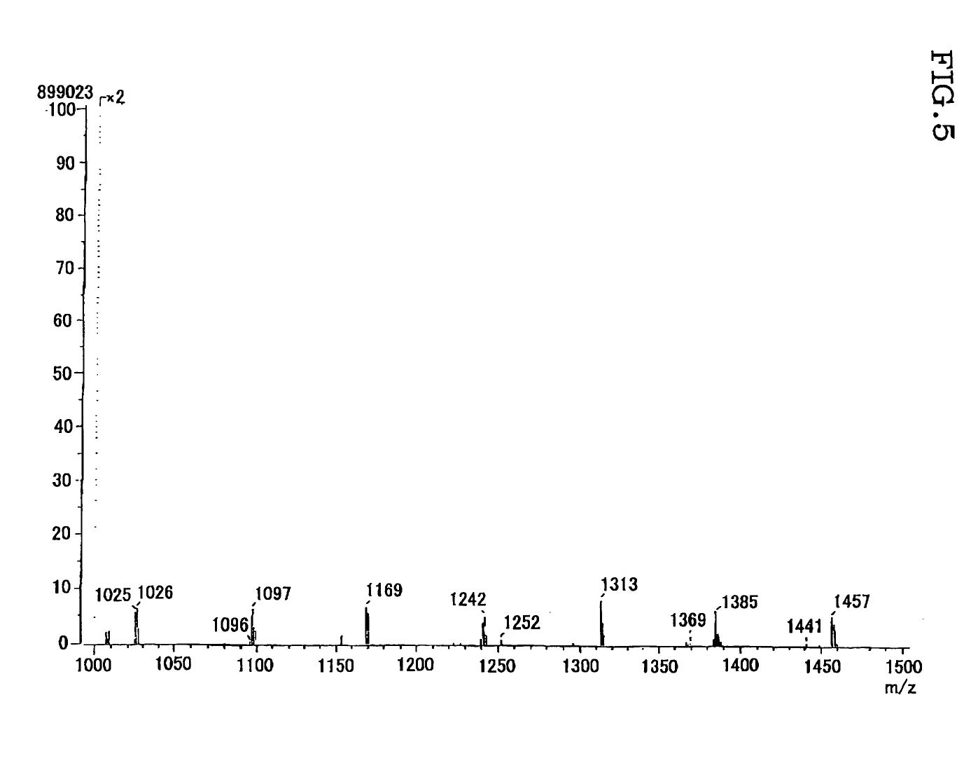

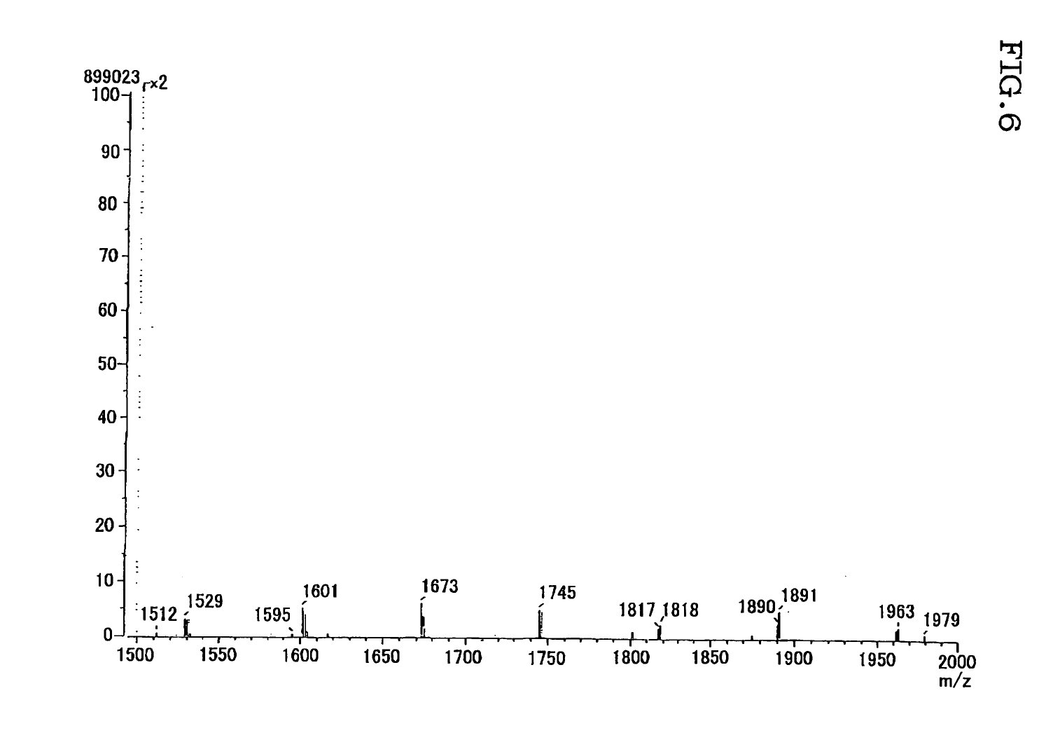

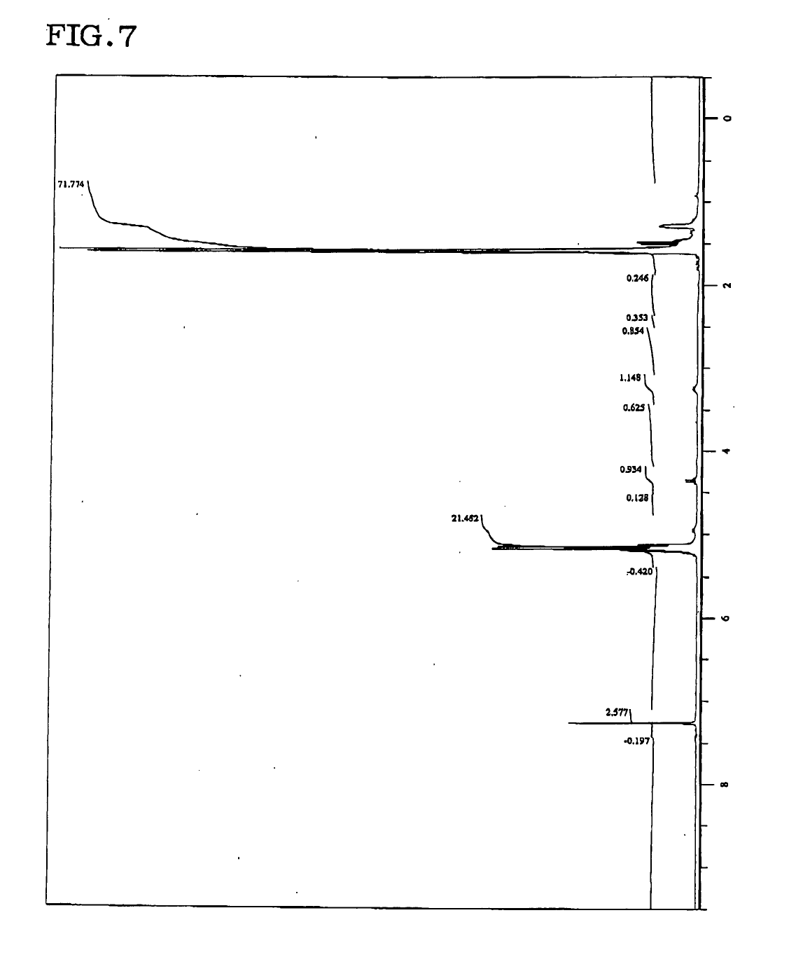

The physical data of the obtained product are shown in FIGS. 1 to 7. From the FABMS and NMR data shown in FIGS. 1 to 7, it was confirmed that a 3-mer to 21-mer cyclic lactic acid oligomer and a 3-mer to 27-mer linear lactic acid oligomer were present in the solid product.

### Test Example 1

(Material and Method)

Male mouse models (CBA/J) of spontaneous alveolar epithelial hyperplasia were employed as test animals. These mice develop alveolar epithelial hyperplasia 12 to 15 weeks after birth. Accordingly, they were raised with normal feeds until 19 weeks after birth. They were then divided into: the group to which feeds containing 0.01% CPL (prepared in Production Example 1) was administered; the group to which adriamycin (ADM) was administered; the group to which feeds containing 0.01% CPL and adriamycin were administered; and the group that did not experience administration. CPL was mixed with powder feeds (CE-2). The daily dosage of adriamycin (ADM) was 0.1 mg/kg per mouse, and administration was made intraperitoneally once a day for three consecutive days, followed by drug withdrawal for 11 days. This procedure was designated as constituting a single course, and eight courses thereof were conducted.

At the time of ADM administration, the body weights of mice were measured and their general conditions were observed. In particular, the occurrence of hair loss was carefully observed. The mice were subjected to autopsy 36 weeks or 37 weeks after birth (with an administration duration of 17 weeks or 18 weeks), and samples were recovered. Tissue blocks were obtained from 3 sites each of the extirpated right and left lungs (6 sites in total), the recovered tissue fragments were fixed, fragments embedded in hydrophilic methacrylate resins were subjected to H-E staining, and the ranges of alveolar epithelial hyperplasia were compared.

(Results)

(1) Change in Body Weight

Body weights of all individuals were measured every other week, and no significant differences were observed among groups.

(2) Effects of Alleviating Side Effects

Hair loss was observed around the eyeballs and the skin of the upper lips of 90% of individuals (9 out of 10 cases) in the group to which ADM alone had been administered. Such hair loss was not observed in any individual in the group to which CPL alone had been administered or the group to which ADM and CPL had been administered in combination. This indicates that CPL had the effects of suppressing side effects caused by ADM (Table 1).

TABLE 1
Comparison of side effects (hair loss) development

| | Number of indi- | |
|---|---|---|
| | viduals under- | Number of indi- |
| | going hair loss | viduals under- |
| | around the | going hair loss |
| | eyeballs | at the upper lip |
| Group to which CPL had been | 0 | 0 |
| administered (n = 7) | | |
| Group to which CPL and ADM | 0 | 0 |
| had been administered (n = 8) | | |
| Group to which ADM had | 9 | 9 |
| been administered (n = 10) | | |
| Group that did not experience | 0 | 0 |
| administration (n = 10) | | |

(3) Effects of Inhibiting Hyperplasia

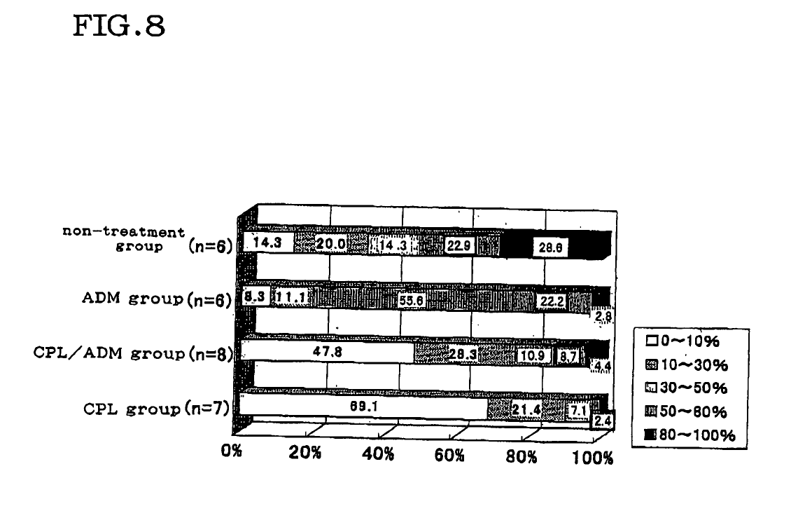

Tissue sections of the tissue samples of the right and left lungs extirpated from the individuals (from 3 sites each, 6 sites in total) were prepared, the range of hyperplasia in the sections was classified into five levels, and groups were compared with each other. The results are shown in FIG. 8. As is apparent from the results shown in FIG. 8, the sections with rates of hyperplasia development of less than 10% accounted for 69.1% of the whole in the group to which CPL had been administered. This was approximately 8.3 times as high as that (8.3%) of the group to which ADM had been administered. Similarly, such sections accounted for 47.8% of the group to which combined administration had been made (the group to which CPL and ADM had been administered). This ratio was approximately 5.8 times as high as that of the group to which ADM alone had been administered.

The above results indicate that the effect of CPL for inhibiting hyperplasia was superior to that of AMD and that the combined administration also enhanced the effect of ADM.

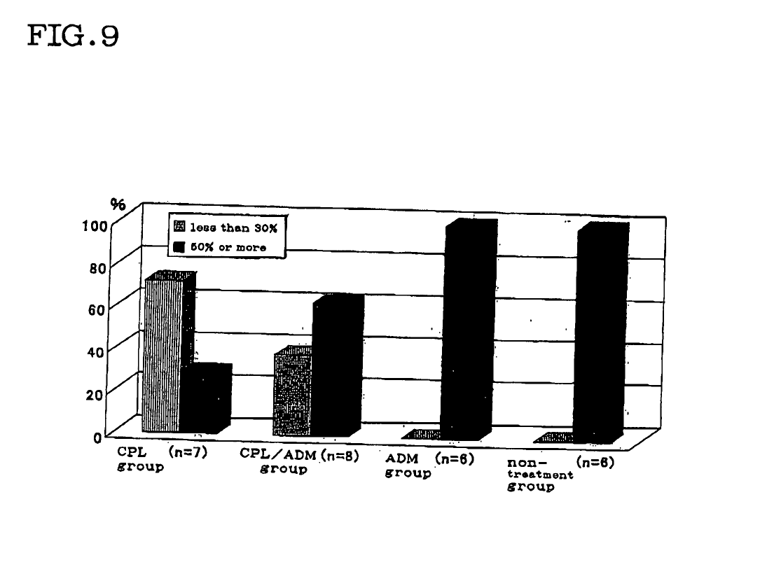

The range of hyperplasia development in each individual was compared. This comparison revealed that the number of individuals with a range of development of less than 30% was 5 out of 7 individuals (71.4%) in the group to which CPL had been administered, and that of the group to which combined administration had been made (the group to which CPL and ADM had been administered) was 3 out of 8 individuals (37.5%). In the case of the group to which ADM had been administered, sections with rates of hyperplasia development of 50% or more were observed in all individuals, as was the case for the group that did not experience administration (FIG. 9).

(Conclusions)

The antitumor effect of CPL was compared with that of ADM, employing the effect of inhibiting alveolar epithelial hyperplasia as an indicator. As a result, the effect of CPL to inhibit hyperplasia was found to be higher than that of ADM under the administration conditions of this example.

Hair loss was observed in 90% of the individuals in the group to which ADM had been administered, which indicates that side effects of ADM had developed. In contrast, hair loss was not observed in any individual of the group to which CPL had been administered, and no abnormality that could be a side effect was observed in terms of appearance.

CPL was administered to the test group to which ADM had been administered, and the effects thereof were examined. As a result, CPL was found to enhance the effects of ADM to inhibit tumors and to inhibit hair loss that had occurred in the group to which ADM had been administered.

### Test Example 2

When several mice are raised in the same cage, examples of possible stressors include overcrowded conditions and a case where a dominant male is present in the group. Under such conditions, hair loss and whisker loss are known to occur. In order to inspect the influence of the overcrowded conditions, 13 groups each consisting of 5 individuals (the group to be raised under normal conditions) and 3 groups each consisting of 10 individuals (the group to be raised under overcrowded conditions) (the number of individuals is 2 times of that of the normal condition) were prepared, and those groups were independently raised in the cages of the same size (depth: 41.5 cm; width: 26 cm; height: 24.5 cm).

Under overcrowded conditions, hair loss and whisker loss were observed in all individuals in 2 out of 3 cages, and hair loss and whisker loss were observed in 9 out of 10 individuals in 1 out of 3 cages. In the case of the groups that had been raised under normal conditions, hair loss and whisker loss were observed in 4 out of 5 individuals in 3 out of 13 cages, no change was observed in 9 out of 13 cages, and hair loss and whisker loss were observed in 1 out of 5 individuals in 1 out of 13 cages (Table 2).

TABLE 2
Occurrence of hair loss and whisker loss in a group that
had been raised under overcrowded conditions and in a
group that had been raised under normal conditions

| | Hair loss and whisker loss development | | | |
|---|---|---|---|---|
| | All | Excluding 1 | Only 1 | |
| | individuals | mouse | mouse | none |
| Group that had been | 2 | 1 | 0 | 0 |
| raised under overcrowded | | | | |
| conditions (3 cages) | | | | |
| Group that had been | 0 | 3 | 1 | 9 |
| raised under normal | | | | |
| conditions (13 cages) | | | | |

### Test Example 3

Subsequently, mice that did not develop hair loss or whisker loss in the group that had been raised under normal conditions were identified. This mouse was removed from a cage containing only one such mouse and it was transferred to the cage in which hair loss or whisker loss had not been observed in all individual in a group that had been raised under normal conditions. As a result, individuals in the cage to which the aforementioned mouse had been transferred developed hair loss and whisker loss. In contrast, hair growth and whisker growth were observed in individuals in the-cage where the aforementioned mouse had originally resided. Based on this phenomenon, when only 1 mouse did not develop hair loss or whisker loss in a cage, this individual was designated as the dominant male.

### Test Example 4

Based on the results attained in Test Examples 2 and 3, hair loss and whisker loss were observed under overcrowded conditions or in the presence of a dominant male. Even when the number of individuals to be raised in the same cage was reduced, hair loss or whisker loss was found to occur in the presence of the dominant male. Accordingly, only the cage in which the dominant male was not present among the group that had been raised under normal conditions was selected, and the effects of CPL for alleviating side effects of an antitumor agent were examined.

(1) Method

Mouse models of spontaneous cancer (CBA/J, 5 males) were raised in a cage until the 54th week, and it was confirmed that hair loss, whisker loss or the like did not occur. Thereafter, they were divided into: the group to which an antitumor agent (adriamycin, ADM) had been administered; the group to which CPL-containing feeds had been administered; and the group to which ADM had been administered in combination with CPL, and these groups of mice were raised. The daily dosage of ADM was 0.2 mg/kg per mouse, and administration was made intraperitoneally once a day for 3 consecutive days, followed by drug withdrawal for 11 days. This procedure was designated as constituting a single course and then continued. CPL was mixed with feeds (CE-2) in amounts of 0.01% thereof.

(2) Results

After the initiation of the experiment, whisker loss and hair loss at the upper jaw were observed on the 6th week (at the third course) in the group to which ADM had been administered (Table 3), and they were continuously raised until the 18th week. It was found that hair loss and whisker loss did not occur in the group to which combined administration had been made and in the group to which CPL had been administered. Accordingly, CPL was found to have the effect of inhibiting side effects of an antitumor agent.

TABLE 3
Side effect development in each group

| | Hair loss and whisker loss |
|---|---|
| | (number of individuals) |
| Group to which ADM had been | 4 |
| administered (n = 10) | |
| Group to which ADM and CPL had been | 0 |
| administered (n = 9) | |
| Group to which CPL had been | 0 |
| administered (n = 10) | |
| Group that did not experience | 0 |
| administration (n = 10) | |

### INDUSTRIAL APPLICABILITY

The agent for suppressing side effects of an antitumor agent and the agent for inhibiting hair loss according to the present invention can be used for inhibiting side effects such as hair loss caused by the administration of antitumor agents. The agent for suppressing side effects of an antitumor agent according to the present invention can enhance the antitumor effect of antitumor agents. Further, since a mixture of poly lactic acids that is used as an active ingredient in the present invention is a less-condensed form of lactic acid derived from a biological component, the mixture of poly lactic acids has high biocompatibility and few side effects.

1. An agent for suppressing side effects of an antitumor agent which comprises a mixture of cyclic and/or straight chain poly lactic acids having a condensation degree of 3 to 20.

2. An agent for inhibiting hair loss which comprises a mixture of cyclic and/or straight chain poly lactic acids having a condensation degree of 3 to 20.

3. The agent according to claim 1 which is used for inhibiting hair loss caused by the use of antitumor agents.

4. The agent according to claim 1 wherein the lactic acid, which is a repeating unit in polylactic acid, is substantially comprised of L-lactic acid.

5. The agent according to claim 1 wherein the mixture of cyclic and/or straight chain poly lactic acids having a condensation degree of 3 to 20 is a mixture of polylactic acids that is produced by polymerizing lactide in the presence of the compound represented by formula (3): Me-N(R¹)(R²) wherein Me represents an alkali metal and R¹ and R² each independently represent an aliphatic group or an aromatic group.

6. The agent according to claim 5 wherein Me represents lithium.

7. The agent according to claim 5 wherein R¹ and R² each independently represent an alkyl group having 1 to 6 carbon atoms.

8. The agent according to claim 5 wherein Me represents lithium, and R¹ and R² represent an isopropyl group.

9. The agent according to claim 1 wherein the mixture of cyclic and/or straight chain poly lactic acids having a condensation degree of 3 to 20 is a substantially cyclic poly lactic acid mixture.

10. Food and drink for suppressing the side effects of an antitumor agent or inhibiting hair loss, which comprises the agent according to claim 1.
